(19) United States
(12) Patent Application Publication
(10) Pub. No.: US 2016/0303013 A1
(43) Pub. Date: Oct. 20, 2016

(54) METHOD OF TREATING HAIR

(71) Applicant: CONOPCO , INC., D/B/A UNILEVER, Englewood Cliffs, NJ (US)

(72) Inventors: Andrew Malcolm MURRAY, Parkgate, Sheshire (GB); Prem Kumar Cheyalazhagan PAUL, Spital, Wirral (GB)

(73) Assignee: Conopco, Inc., d/b/a UNILEVER, Englewood Cliffs (GB)

(21) Appl. No.: 15/036,445

(22) PCT Filed: Nov. 19, 2014
(86) PCT No.: PCT/EP2014/075000

§ 371 Date: May 13, 2016

(30) Foreign Application Priority Data
Nov. 21, 2013 (EP) 13193934.0

(51) Int. Cl. A61K 8/365; A45D 7/04; A61K 8/04; A61K 8/41; A61Q 5/06; A61Q 5/12

(52) U.S. Cl. CPC A61K 8/365; A61Q 5/06; A61Q 5/12; A61K 8/042; A61K 8/416; A45D 7/04; A61K 2800/596

(57) ABSTRACT

A method of styling hair by applying from 3 to 15 g di-or tri-carboxylic acid.

The invention relates to a method of styling hair.

Permanent hair straightening compositions that are on the market are based on chemical treatment of the hair in a two-step process using thiol- or hydroxide-based reducing agents followed by a neutralisation or oxidation step. Such systems have various negatives associated with them; in that the process itself is difficult to conduct, in many instances this straightening process is undertaken by a qualified hairdresser in a professional salon. Furthermore the straightening process damages the hair, has an unpleasant odor and can cause irritation to the scalp.

Surprisingly we have found that hair can be styled without causing damage, without using hair irons and the hair remains styled even after subsequent washing.

Accordingly, the present invention provides a method of styling hair by applying from 3 to 15 g di- or tri-carboxylic acid.

Preferably, the method comprises applying the di-or tri-carboxylic acid as an aqueous solution or aqueous suspension.

Preferably and where the method comprises applying an aqueous solution or suspension the pH of the solution or suspension is from 1 to 3, more preferably from 1.0 to 3.0.

Preferably, where the method comprises applying an aqueous solution or suspension of carboxylic acid to the hair the concentration of carboxylic acid is from 2-25% wt. of the solution or suspension, more preferably from 3-20 wt % and most preferably from 4-8 wt% .

Preferably, the method comprises applying the di- or tri-carboxylic acid to dry hair.

By ‘dry hair’ is meant that the amount of free water disposed on the cuticle has been substantially removed by towelling or evaporation such that it constitutes no more than 25% wt. of the hair fibre as a whole.

This means that the hair has not been washed or actively wetted, such as by shampooing, conditioning, rinsing or otherwise treating with an aqueous composition in the preceding 2, preferably 3 hours and has been permitted to acclimatise to atmospheric conditions.

In such circumstances there is substantially no free water on the hair fibre which interferes with the adsorption of the hair treatment composition on application.

Preferably, the method comprises applying a hair treatment composition comprising the carboxylic acid and preferably this hair treatment composition comprises conditioning materials. Preferred conditioning materials include cationic surfactants, silicones, fatty alcohols and mixtures thereof.

Preferably, the hair treatment composition comprises cationic surfactant having the formula N+R¹R²R³R⁴ wherein R¹, R², R³ and R⁴ are independently (C₁ to C₃₀) alkyl or benzyl. Preferably, one, two or three of R¹, R², R³ and R⁴ are independently (C₄ to C₃₀) alkyl and the other R¹, R², R³ and R⁴ group or groups are (C₁-C₆) alkyl or benzyl. More preferably, one or two of R¹, R², R³ and R⁴ are independently (C₆ to C₃₀) alkyl and the other R¹, R², R³ and R⁴ groups are (C₁-C₆) alkyl or benzyl groups. Optionally, the alkyl groups may comprise one or more ester (—OCO— or —COO—) and/or ether (—O—) linkages within the alkyl chain. Alkyl groups may optionally be substituted with one or more hydroxyl groups. Alkyl groups may be straight chain or branched and, for alkyl groups having 3 or more carbon atoms, cyclic. The alkyl groups may be saturated or may contain one or more carbon-carbon double bonds (eg, oleyl). Alkyl groups are optionally ethoxylated on the alkyl chain with one or more ethyleneoxy groups.

Suitable cationic surfactants for use in conditioner compositions according to the invention include cetyltrimethylammonium chloride, behenyltrimethylammonium chloride, cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, dihydrogenated tallow dimethyl ammonium chloride (eg, Arquad 2HT/75 from

Akzo Nobel), cocotrimethylammonium chloride, PEG-2-oleammonium chloride and the corresponding hydroxides thereof. Mixtures of any of the foregoing materials may also be suitable. A particularly useful cationic surfactant for use in conditioners according to the invention is cetyltrimethylammonium chloride, available commercially, for example as GENAMIN CTAC, ex Hoechst Celanese.

Another particularly useful cationic surfactant for use in conditioners according to the invention is behenyltrimethylammonium chloride, available commercially, for example as GENAMIN KDMP, ex Clariant.

Preferably the cationic surfactant would be present at from 0.1 to 10% wt., more preferably from 0.5 to 7.5% wt. and most preferably from 0.5 to 5% wt. of any hair treatment composition used in accordance with the method of the invention.

Preferably, the treatment composition for use in the method of the invention comprises from 0 to 0.1% wt. and is more preferably free from an amidoamine corresponding to the general formula (I):

R1CONH(CH2)mN(R2)R3

in which R¹ is a hydrocarbyl chain having 10 or more carbon atoms,

R² and R³ are independently selected from hydrocarbyl chains of from 1 to 10 carbon atoms, and m is an integer from 1 to about 10; and

Notable amidoamines include stearamido-propyldimethylamine, stearamidopropyldiethylamine, stearamidoethyldiethylamine, stearamidoethyldimethylamine, palmitamidopropyldimethylamine, palmitamidopropyl-diethylamine, palmitamidoethyldiethylamine, palmitamidoethyldimethylamine, behenamidopropyldimethyl-amine, behenamidopropyldiethylmine, behenamidoethyldiethyl-amine, behenamidoethyldimethylamine, arachidamidopropyl-dimethylamine, arachidamidopropyldiethylamine, arachid-amidoethyldiethylamine, arachidamidoethyldimethylamine, and mixtures thereof.

Such amidoamines are typically included with an acid which protonises the amine to form a cationic surfactant.

Preferably, the di- or tri-carboxylic acid is applied to the hair and left for at least 5 minutes, preferably at least 10 minutes, more preferably at least 15 minutes and most preferably at least 20 minutes before being rinsed off. Preferably the acid is rinsed off 90 minutes after application, more preferably 60 minutes and most preferably 40 minutes after application.

Preferably, the method is conducted without the addition of heat in the form of hair straighteners or irons. Accordingly, the method is conducted at from 15 to 45° C., more preferably from 20 to 30° C.

Preferred di-or tri-carboxylic acids include aconitic acid, tricarballylic acid, malonic acid, tartaric acid, citric acid and mixtures thereof. The most preferred acids are citric and aconitic and all references to preferred features of the invention are applicable to each and every acid mentioned herein and especially for citric acid and aconitic acid.

Preferably by styling the hair is meant straightening, retaining curl or providing body.

In a second aspect there is provided a method for styling the hair by applying from 3 to 15g di- or tri-carboxylic acid which is provided in a hair treatment composition of volume ranging from 100 to 300 ml, more preferably, from 150 to 250 ml.

The following non-limiting examples further illustrate the preferred embodiments of the invention. All percentages referred to in the examples and throughout this specification are by weight based on total weight unless otherwise indicated.

### EXAMPLE

The citric acid is made separately and buffered to a pH of 2.2-2.3 with sodium hydroxide before adding to a conditioning composition base comprising the remaining materials, notably the cationic surfactant and fatty alcohol which together form the conditioning gel phase.

| Trade Name | % w | % w | % w | % w |
|---|---|---|---|---|
| Water | To 100 | To 100 | To 100 | To 100 |
| Natrosol | — | — | — | 0.1 |
| Behentrimmonium chloride (70%) | 4.3 | 0.5714 | 1.1428 | 0.5714 |
| Cetearyl alcohol | 6 | 1.5 | 3 | 1.5 |
| Dimethylacrylamide/ | — | — | — | 1.5 |
| Ethyltrimonium Chloride | | | | |
| Methacrylate Copolymer | | | | |
| (and) Propylene Glycol | | | | |
| Dicaprylate/Dicaprate (and) | | | | |
| PPG-1 Trideceth-6 (and) | | | | |
| C10-C11 Isoparaffin* | | | | |
| Aminosilicone (70%) | 1.428 | 1.428 | 1.428 | 1.428 |
| Preservative (70%) | 0.35 | 0.35 | 0.35 | 0.35 |
| Perfume | 0.6 | 0.6 | 0.6 | 0.6 |
| Citric acid (25% wt aq) | 20 | 20 | 20 | 20 |
| NaOH (50%) | 0.91 | 0.91 | 0.91 | 0.91 |
| EDTA | 0.05 | 0.05 | 0.05 | 0.05 |

*Tinovis CD ex BASF

The following data supports a finding that the straightening achieved through applying to the hair a hair treatment composition as described in the above table is durable through multiple washes.

| | Volume [mm²] | | | | |
|---|---|---|---|---|---|
| Stage | A | B | C | D | E |
| Before | 15474.73 | 17214.53 | 16720.19 | 17085.74 | 16283.36 |
| treatment | | | | | |
| After | 14914.97 | 10919.82 | 10791.08 | 11439.89 | 11541.37 |
| treatment | | | | | |
| AW1 | 14978.74 | 10860.80 | 12622.58 | 12480.31 | 13421.44 |
| AW3 | 14848.99 | 11538.96 | 13427.67 | 13212.13 | 14161.78 |
| AW5 | 14882.07 | 11278.24 | 13206.93 | 13375.39 | 13621.05 |
| AW10 | 14886.58 | 11838.58 | 12364.28 | 13224.90 | 14097.57 |
| AW15 | 13927.47 | 11188.64 | 12216.97 | 13636.92 | 14520.90 |
| AW20 | 14218.68 | 13249.26 | 15034.80 | 15348.22 | 15422.16 |

‘AW X’ means after X washes.

| | Percentage Volume | | | | |
|---|---|---|---|---|---|
| Stage | A | B | C | D | E |
| Before | 100 | 100 | 100 | 100 | 100 |
| treatment | | | | | |
| After | 96.38 | 63.43 | 64.54 | 66.96 | 70.88 |
| treatment | | | | | |
| AW1 | 96.79 | 63.09 | 75.49 | 73.05 | 82.42 |
| AW3 | 95.96 | 67.03 | 80.31 | 77.33 | 86.97 |
| AW5 | 96.17 | 65.52 | 78.99 | 78.28 | 83.65 |
| AW10 | 96.20 | 68.77 | 73.95 | 77.40 | 86.58 |
| AW15 | 90.00 | 65.00 | 73.07 | 79.81 | 89.18 |
| AW20 | 91.88 | 76.97 | 89.92 | 89.83 | 94.71 |

1. A method of styling hair by applying from 3 to 15 g di- or tri-carboxylic acid wherein the method comprises applying an aqueous solution or suspension of the di- or tri- carboxylic acid to the hair and in which the acid is left on the hair for at least 10 minutes and less than 90 minutes.

2. A method according to claim 1 in which the acid is left on the hair for at least 15 minutes and less than 90 minutes.

3. A method according to claim 1 or 2 in which the acid is present in an aqueous solution to aqueous suspension.

4. A method according to any preceding claim which is conducted at room temperature.

5. A method according to any preceding claim wherein the acid is applied to dry hair.

6. A method according to any preceding claim wherein styling constitutes straightening, providing body or retaining curl.

7. A method according to claim 3 wherein the pH of the solution or suspension is from 1.0 to 3.0.

8. A method according to any preceding claim wherein the acid forms part of a hair treatment composition which comprises a mono-or di-alkyl quaternium salt.

9. A method for styling hair comprising:
i) applying to hair an aqueous solution or suspension comprising from 3 to 15 g di- or tri-carboxylic acid and having a pH of from 1 to 3;
ii) leaving the product on the hair for from 5 to 90 minutes;
iii) rinsing the product from the hair; and styling.

10. A method for durably styling the hair by following a method according to any preceding claim.
